(19) United States
(12) Patent Application Publication
(10) Pub. No.: US 2002/0151058 A1
(43) Pub. Date: Oct. 17, 2002

(54) VECTOR CONSTRUCTION BY HOST CELL-MEDIATED RECOMBINATION

(76) Inventors: EDWARD L. PERKINS, SUNNYVALE, CA (US); STUART M. TUGENDREICH, MOUNTAIN VIEW, CA (US)

Correspondence Address: ROBINS & PASTERNAK LLP, 545 MIDDLEFIELD ROAD, SUITE 180, MENLO PARK, CA, 94025, US

(21) Appl. No.: 09/336,421

(22) Filed: Jun. 17, 1999

Related U.S. Application Data
(60) Provisional application No. 60/089,676, filed on Jun. 17, 1998.

(51) Int. Cl. C12N 15/85

(52) U.S. Cl. 435/455

(57) ABSTRACT

Described are methods and materials which enable rapid, efficient, and scalable cloning of one or more specific target nucleic acid molecules into suitable expression vectors without the need for performing an in vitro ligation step. The invention takes advantage of gap repair mechanisms to produce the desired expression vector(s) in host cells capable of mediating intermolecular homologous recombination. Specific target nucleic acids are cloned by producing expression vector intermediates containing two sequence-specific recombination regions, each of which is substantially homologous to a specific recombination sequence flanking the desired target nucleic acid, through a nucleic acid amplification process using primers which each include a different 5′ sequence-specific recombination sequence which lacks complementarity with the base vector and a different 3′ priming portion substantially complementary to a primer binding site in the base vector.

### PRIOR APPLICATION

This application claims priority from U.S. provisional application No. 60/089676, filed Jun. 17, 1998, incorporated herein by reference in full.

### FIELD OF THE INVENTION

This invention relates generally to the fields of molecular biology and vector construction. More particularly, the invention relates to cloning methods wherein two or more nucleic acids are joined into a desired construct by in vivo recombination.

### BACKGROUND OF THE INVENTION

Techniques for manipulating nucleic acid molecules are of critical importance in biotechnology. The discovery of restriction endonucleases, nucleic acid polymerases, ligases, and other enzymes, and the cloning of many of the genes encoding these proteins, has facilitated the rapid development and commercialization of this technology. However, most modem cloning techniques are suitable for relatively small nucleic acids. Moreover, high throughput, or massively parallel, cloning techniques generally have not been developed.

To overcome the difficulties attendant with the cloning of large nucleic acid molecules, specialized nucleic acid vectors have been developed. These vectors, frequently referred to as “Yeast Artificial Chromosomes,” or “YACs,” enable one to clone large chromosomal fragments (e.g., one million or more nucleotide base pairs), and have aided in the characterization of large genomes. See, for example, Resnick et al., WO98/01573. Similar systems known as “Bacterial Artificial Chromosomes” (“BACs”) have recently been developed for propagating large, cloned fragments in bacteria. However, the upper size limit of nucleic acid molecules that can be cloned into BACs is much lower, typically less than about 150 kilobases (kb).

Irrespective of the vector employed, traditional nucleic acid cloning methods employ at least one in vitro ligation step, where the vector nucleic acid is ligated to one or more insert nucleic acids by a DNA ligase, although ligation is sometimes omitted if single stranded regions having sufficient complementarity exist on the vector and insert nucleic acids, such that stable, double-stranded hybrids can form prior to transformation into the desired host cell. After uptake, the strands would be joined by the host cell's ligation machinery. See Oliner et al., Nucleic Acids Res. (1993) 22:5192-97.

More recently, techniques have been developed to take advantage of a cell's ability to perform “recombination” between double-stranded nucleic acids sharing regions of homology. FIG. 1 illustrates a single recombination event between two nucleic acid molecules 10 and 11 at their respective regions of homology 2, to produce nucleic acid molecule 15 which contains both sequence A (1) and sequence B (4) following co-transformation of nucleic acid molecules 10 and 11 and host cell-mediated homologous recombination via a single event. One example is the Transformation-Associated Recombination (TAR) approach reported in PCT WO98/01573. TAR is based on the use of a vector having two regions of homology with an insert nucleic acid, which regions are both non-specific or one is specific and one is non-specific. Upon introduction into an appropriate host cell, homologous recombination between the homologous regions of the vector and insert produce a completed vector, which is linear or circular, depending on the nucleic acids used. For cloning large fragments, the vector typically contains two copies of the desired region of homology, e.g., an Alu sequence when cloning large human DNA fragments. For cloning specific nucleic acids, the vector contains one sequence-specific area of homology (e.g., a known sequence for a specific cDNA, as may be derived from an Expressed Sequence Tag, or EST) and one non-specific region of homology, i.e., the sequence may be known, but it is not specific for a particular nucleic acid.

While useful in some contexts, TAR cloning requires that a different vector be generated for each nucleic acid to be cloned. Moreover, sequence-specific targeting of particular target nucleic acids is not provided due to the absence of two sequence-specific recombination regions on the vector, nor is TAR cloning readily adaptable to high throughput formats. In addition, when the vector carries two of the same or largely homologous recombination sequences, vector-only constructs can be produced by recombination between the vector-carried recombination sequences.

Oliner et al., have reported a procedure similar to TAR for use in Escherichia coli, called in vivo cloning (IVC). In IVC, a particular nucleic acid sequence is amplified via PCR. The resulting PCR products contain ends having overlapping regions of homology with a desired vector, both of which were co-transformed into a recombination-competent E. coli strain.

### SUMMARY OF THE INVENTION

Despite such advances, a need still exists for more rapid, efficient, and scalable cloning techniques. This invention provides a solution for such needs, specifically by providing methods and materials wherein amplification of a base vector using different primers allows the rapid, efficient production of multiple intermediate vectors that differ only in the two sequence-specific recombination regions they carry, thereby allowing different, specific target nucleic acids to be efficiently cloned.

It is an object of this invention to provide methods and materials useful for rapid, efficient, and scalable cloning of genes and other target nucleic acids of interest. One aspect of the invention relates to methods of making expression vectors by introducing into recombination-competent host cells an expression vector intermediate and a population of nucleic acid molecules expected or suspected to contain a target nucleic acid. The expression vector intermediate typically is linear and comprises an autonomous propagation sequence, a first terminus having its first sequence-specific recombination region, and a second terminus having a second sequence-specific recombination region. Through homologous recombination events catalyzed by enzymes expressed in the host cell, circularized expression vectors comprising the target nucleic acid can be produced. In certain embodiments, the recombination-competent host cell is a eukaryotic cell, for example, a mammalian, fungal, plant, or insect cell. Yeast host cells, particularly strains of Saccharomyces cerevisiae, are preferred host cells. In other embodiments, the recombination-competent host cell is a prokaryotic cell, for example, a recombination-competent strain of E. coli.

Another aspect of the invention is a method for producing a plurality of vectors, by providing a first polynucleotide comprising an autonomous propagation sequence (APS) flanked by first and second homologous recombination sequences (which sequences do not recombine with each other), and a plurality of second polynucleotides, each second polynucleotide comprising a target sequence (or potential target sequence) flanked by third and fourth homologous recombination sequences which are capable of recombination with said first and second homologous recombination sequences, and transforming a replication-competent host cell with said polynucleotides to provide a plurality of recombined vectors, each comprising an APS and a target sequence. In a subgenus of the invention, said first polynucleotide and/or said second polynucleotides further comprise selectable markers. In another subgenus of the invention, said first polynucleotide or said second polynucleotides further comprise a promoter, such that the vectors that result are expression vectors. In another subgenus of the invention, the resulting vector comprises homologous recombination sequences flanking said target sequence that are sufficient for homologous recombination with the host cell genome. In another subgenus of the invention, said first and/or second polynucleotides are provided by providing a polynucleotide having first and second primer binding sites, and amplifying said polynucleotide by PCR using first and second primers, wherein said primers comprise a sequence complementary to a primer binding site and a homologous recombination sequence, such that the resulting polynucleotides comprise an APS (in the case of the first polynucleotide) and/or a target sequence (in the case of the second polynucleotides) flanked by primer binding sites, in turn flanked by homologous recombination sites. In each case, the resulting transformed host cell can then be screened for the presence of a vector, either by the presence of a marker, or by the absence of a negative marker (a marker deleterious to the host cell that is excised during homologous recombination), or by the presence of a phenotype due to the target gene.

Another aspect of the invention is a kit for use in the method of the invention, comprising a base vector comprising an autonomous propagation sequence, a first homologous recombination site, and a second homologous recombination site; and a set of primers for amplifying a target nucleic acid and adding homologous recombination sites to said target nucleic acid.

### BRIEF DESCRIPTION OF THE FIGURES

FIG. 1 depicts schematically a homologous recombination event between two nucleic acids having a region of homology.

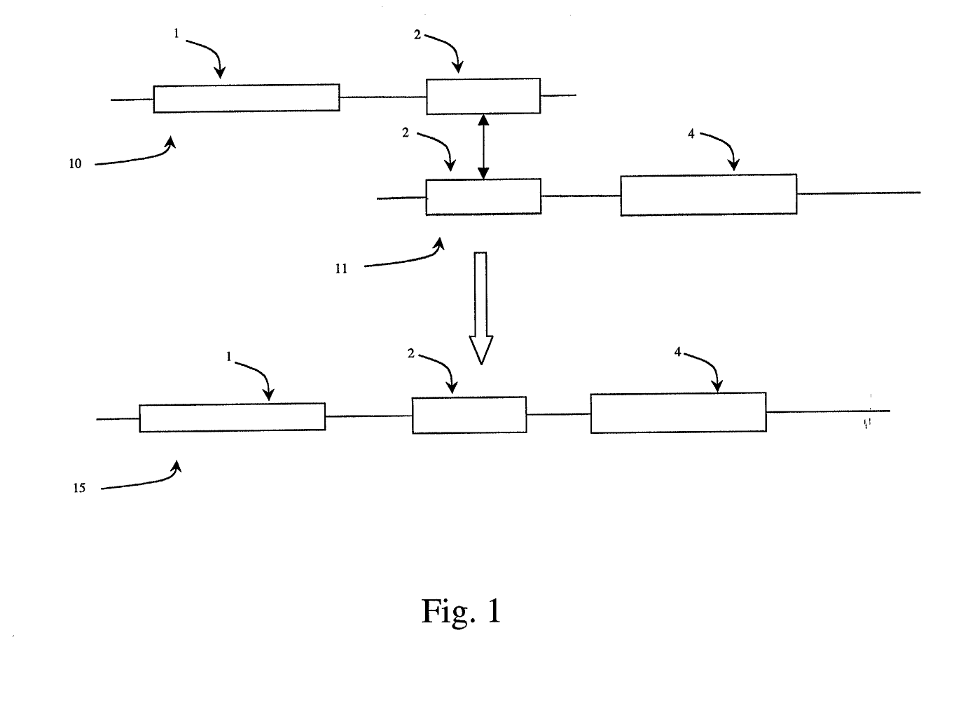

FIG. 2 depicts schematically a double homologous recombination, between two nucleic acids having two separated regions of homology.

FIG. 3 depicts a triple homologous recombination, between a circular plasmid and two linear nucleic acids.

FIG. 4 depicts insertion of a cDNA into a plasmid by homologous recombination.

FIG. 5 is a map of plasmid pARC253-1.

### GENERAL METHOD AND DETAILED DESCRIPTION

Definitions

An “expression vector” is a nucleic acid construct from which expression of one or more genes (i.e., a double-stranded nucleic acid molecule encoding an open reading frame comprising two or more codons) can occur. At a minimum, such a vector will include a nucleotide sequence which allows the vector to be propagated in one or more different host cells and a transcription initiation sequence (e.g., a promoter or other sequence from which an RNA polymerase can initiate transcription of mRNA) operably associated with a gene of interest. While not essential, such vectors typically also include one or more selectable markers and a multiple cloning site comprising two or more restriction endonuclease cleavage sites.

“Host cell” refers to a microorganism into which a mixture of nucleic acids can be introduced by any appropriate means (e.g., transformation, transfection, electroporation, ballistic bombardment, and conjugation). A “recombination competent” host cell is one in which intermolecular homologous recombination can occur. Such-host cells include both prokaryotic and eukaryotic host cells.

A “eukaryotic” host cell is a host cell having a membrane-encapsulated nucleus. Representative examples of eukaryotic host cells include mammalian cells, fungal cells, plant cells, and insect cells. Particularly preferred are fungal host cells such as yeast, particularly strains of Saccharomyces cerevisiae. A “prokaryotic” host cell is one lacking a membrane-enclosed nucleus. Representative examples include bacteria such as, for example, Escherichia coli.

An “autonomous propagation sequence” refers to a nucleotide sequence conferring the ability upon a nucleic acid construct carrying the same (e.g., an expression vector) to be replicated and segregated during multiple cell divisions. Many such sequences are known, and include origins of replication from various bacterial plasmids and extra-chromosomal elements found in various eukaryotic cells, e.g., episomes and 2 micron (2μ) circles found in yeast.

A “sequence-specific recombination region” or “homologous recombination site” refers to a nucleotide sequence which enables homologous recombination between two nucleic acids having substantially the same (greater than about 75% nucleotide sequence homology, preferably greater than about 85% nucleotide homology, and particularly greater than about 95% nucleotide sequence homology) nucleotide sequence in a particular region of two different nucleic acid molecules. When two or more such regions are employed, discrete portions of nucleic acids between the sequence-specific recombination region on one nucleic acid molecule can be homologously recombined into another nucleic acid molecule between the corresponding recombination regions in the second nucleic acid molecule. These regions can be located at anywhere along the length of a nucleic acid molecule. For example, on a linear molecule, they may be located at, or include, the terminal nucleotide base pairs of a nucleic acid molecule. Alternatively, such a region need not include the terminal nucleotide base pairs of a linear nucleic acid, but instead can begin one or more base pairs from, or “internal” to, the terminal base pair of a nucleic acid molecule. Moreover, sequence-specific recombination regions can be any size, so long as they are sufficient in length to enable homologous recombination between two nucleic acids comprising substantially the same nucleotide sequence in this region. Preferably, such regions comprise at least about 15 nucleotides, with a range of about 25 to about 500 nucleotides being more preferred. Given that at least some these sequences are typically included in synthetic oligonucleotide amplification primers used to produce expression vector intermediates, an especially preferred size ranges from about 25 to about 60 nucleotides. Homologous recombination sequences for vector construction are preferably not homologous to the host cell genome.

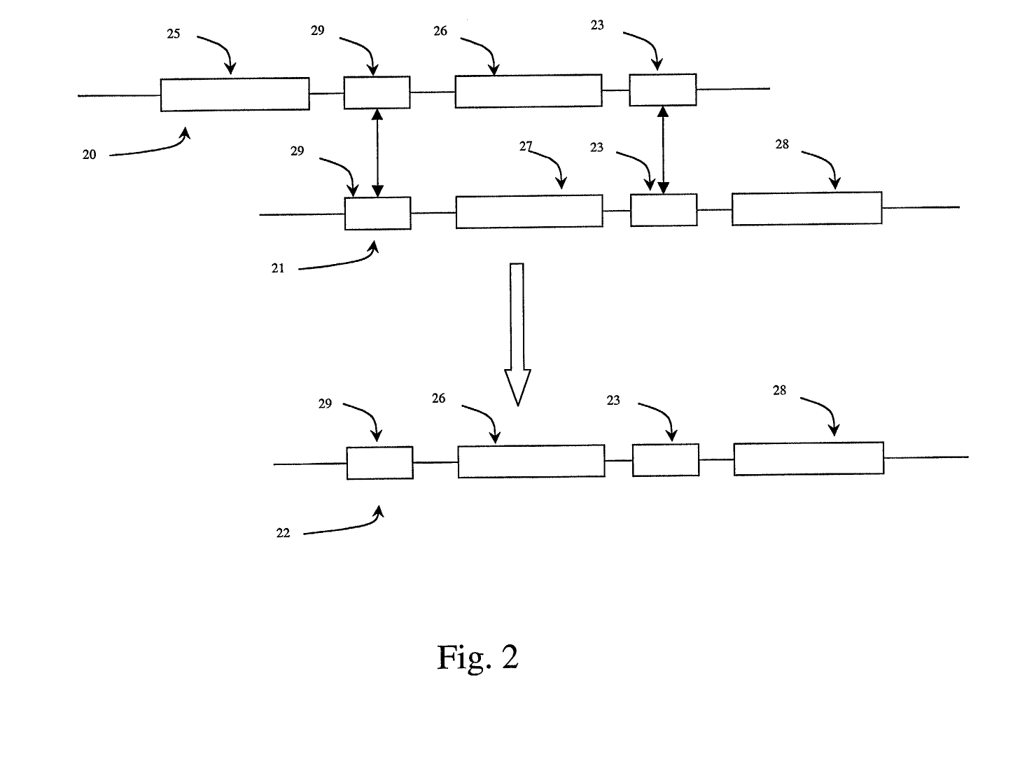

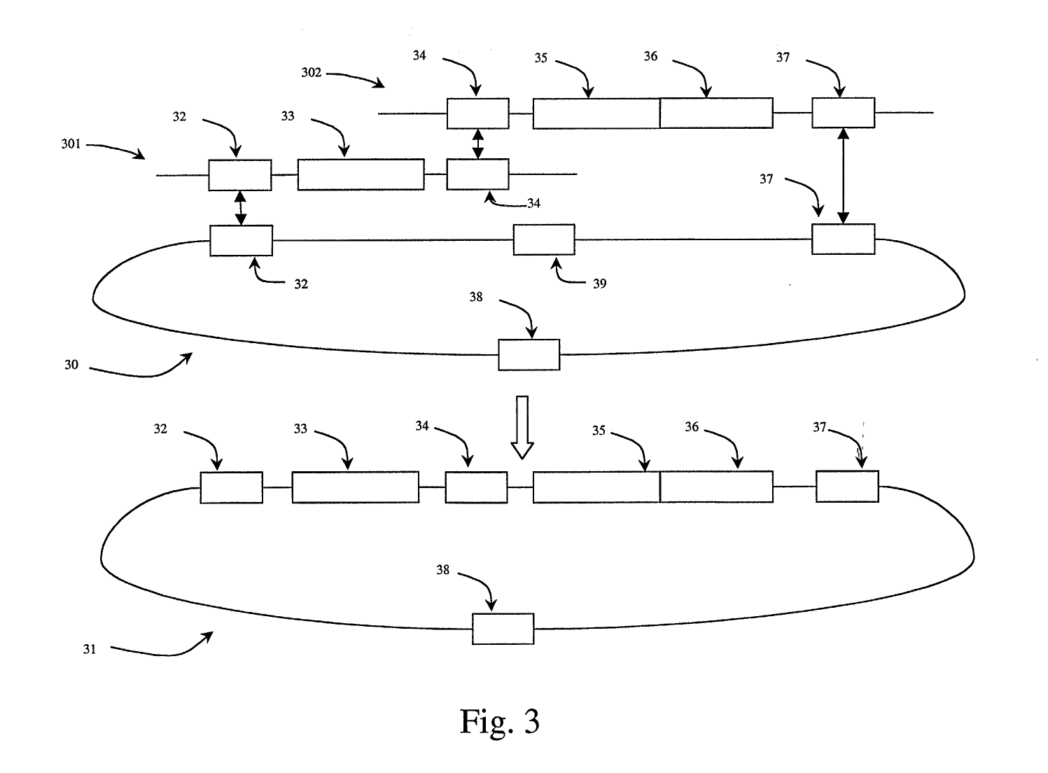

A “contiguous pre-recombination nucleotide sequence” refers to a nucleic acid which is not required to undergo recombination prior to being recombined with an expression vector intermediate according to the invention. For example, polynucleotides 20 and 21 in FIG. 2 are contiguous pre-recombination nucleotides, because each has the homologous recombination sites necessary for recombination to occur. In contrast, a “non-contiguous fragment” refers to a nucleic acid molecule which requires recombination with at least one other fragment before recombination can produce a complete expression vector intermediate according to the invention. For example, polynucleotides 301 and 302 of FIG. 3 are non-contiguous fragments because neither alone is capable of recombining with both homologous recombination sites of polynucleotide 30. Non-contiguous fragments are not required to recombine with each other prior to recombining with the first polynucleotide, but may proceed in any order or simultaneously. Such fragments can include one or more genes of interest carried on one fragment and one or more selection cassettes carried on another fragment. For example, in FIG. 3, sequence 33 can be a promoter while sequences 35 and 36 represent protein coding sequences, or sequence 33 can represent a selectable marker while sequences 35 and 36 represent a promoter and a protein coding sequence, respectively. Homologous recombination between such fragments occurs through “target forming recombination elements” or “homologous recombination sequences” located on each fragment. Such elements comprise a particular type of sequence-specific recombination region, with which they otherwise share the same attributes. Moreover, it is not required that such fragments recombine to form contiguous pre-recombination nucleotide sequence prior to homologous recombination with an expression vector intermediate. In addition, it is understood that both the ultimate target nucleic acid, expression vector intermediate, other nucleic acid construct used in the invention may comprise two or more such fragments, or “non-contiguous pre-recombination nucleotide sequences.”

A “selectable marker” refers any genetic element which, when expressed, confers upon a cell containing such marker the ability to be selected from cells which do not contain or express such marker. Typically, such markers encode drug resistance genes or a protein involved in a pathway for the synthesis of a metabolite necessary for the host microorganism to grow and survive in a media lacking the particular metabolite, ie., a positive selectable marker. Representative examples include genes encoding beta-lactamase, TRP1, and LEU2. On the other hand, “negative” selection can be used, wherein expression of the negative selection marker typically prevents propagation of the host cells under the appropriate conditions thereby allowing selection for cells failing to express the marker, as may result when such a marker causes cells to die or to grow more slowly than those which do not express the marker, or which render then susceptible to drugs or environmental stresses. Examples of such markers include, without limitation, those which encode suppressor tRNAs, the LYS2 and URA3 genes, thymidine kinase, and those which allow color-based selection, for example, beta-galactosidase.

A “population of nucleic acids” refers to a pool of nucleic acids expected or suspected to contain a target nucleic acid, i.e., the nucleic acid molecules desired to be incorporated into the expression vector intermediate via host cell-mediated homologous recombination. Such a population, or pool, can contain only multiple copies of a particular nucleic acid molecule or one or more copies of two more different nucleic acid molecules.

“Amplification” of nucleic acids useful in the practice of this invention, particularly base vectors, can be performed any suitable methods. Such methods include amplification via the polymerase chain reaction (“PCR”) and by strand displacement amplification, ligase chain reaction, and transcription-mediated amplification, each of which can be adapted given the teachings provided herein.

### Description Of The Invention

FIG. 2 illustrates a double homologous recombination event between nucleic acids 20 and 21, each of which contain two regions of homology (23 and 29). Nucleic acid 20 contains two sequences of interest 25 and 26, while nucleic acid 21 comprises sequences 27 and 28. Recombination between nucleic acids 20 and 21 produces nucleic acid 22, which is identical to nucleic acid 21, except that the sequence of nucleic acid 21 between the two regions of homology has been replaced the corresponding region from nucleic acid 20, resulting in the replacement of sequence 27 with sequence 26 and production of nucleic acid 22.

In one aspect of the invention, the target nucleic acid typically comprises a nucleotide sequence having a 5′ terminus capable of homologous recombination with the first sequence-specific recombination region of the expression vector intermediate and a 3′ terminus capable of recombination with the second sequence-specific recombination of the expression vector intermediate. These regions of homology on the target nucleic acid are also considered to be sequence-specific recombination regions. While in some embodiments the target nucleic acid comprises a single, contiguous pre-recombination nucleotide sequence, in other embodiments the target nucleic acid may comprise two or more pre-combination fragments. When only two pre-recombination fragments comprise the target nucleic acid, each fragment will comprise an expression vector intermediate-specific sequence capable of recombining with a sequence-recombination region of the expression vector intermediate. In addition, each fragment will contain a target-forming recombination element capable of homologous recombination with the corresponding target-forming recombination element of the other pre-recombination fragment. The expression vector intermediate-specific sequence and target-forming recombination elements may be at the termini of the pre-recombination fragments, internal to the termini of the fragments, or a combination, wherein, for example, an expression vector intermediate-specific sequence comprises the terminal nucleotide base pairs of a pre-recombination fragment, while the target forming recombination element of the fragment is internal to and does not comprise the other terminus of the pre-recombination fragment. In those embodiments wherein three or more pre-recombination fragments are present, it is understood that the two pre-recombination fragments intended to recombine with the sequence-specific recombination regions of the expression vector intermediate will comprise both an expression vector intermediate-specific sequence and a target forming recombination element, while those pre-recombination fragments which do not comprise expression vector intermediate-specific sequences instead contain two target forming recombination elements to enable recombination to form a contiguous target nucleic acid.

FIG. 3 shows a triple homologous recombination event involving nucleic acids 30, 301, and 302, to produce nucleic acid 31. Nucleic acid 30 represents a base vector comprising an autonomous propagation sequence (APS, 38) and two regions of homology, 32 and 37. In some embodiments, the sequence between 32 and 37 contains a negative selection marker (SM, 39) that is excised from the vector upon recombination, as depicted in FIG. 3. Nucleic acid 301 comprises Sequence 33 between two areas of homology (32 and 34), and additional nucleotide sequences flanking 32 and 34 which are lost during recombination. Nucleic acid 302 also comprises two homology regions, 34 and 37, located on either side of two sequences of interest, Sequences 35 and 36. Nucleic acid 302 also comprises nucleotide sequences flanking 34 and 37 which are lost during recombination.

In some embodiments of this aspect of the invention, the expression vector intermediate comprises a selectable marker, a transcription initiation sequence, and/or a transcription termination sequence. Selectable markers include those which both enable selection of host cells containing a selectable marker, while other embodiments enable selecting cells which do not contain the selectable maker, e.g., the selectable marker is toxic to or reduces host cell growth on the media employed. As will be clear to those in the art, each expression vector according to the invention will preferably contain an autonomous propagation sequence which enables the expression vector to be replicated, propagated, and segregated during multiple rounds of host cell division. The autonomous propagation sequence can be either prokaryotic or eukaryotic, and includes an origin of replication. Preferred embodiments of an expression vector according to the invention include both prokaryotic and eukaryotic autonomous propagation sequences.

The first and second sequence-specific recombination regions of an expression vector intermediate according to the invention comprises a double stranded region of at least about 15 nucleotide-base pairs, although such recombination region can be of any size, so long as it is sufficient to enable homologous recombination between the fragment on which it is carried and another nucleic acid having a substantially homologous region. In preferred embodiments, the sequence-specific recombination regions of the expression vector intermediate and target nucleic acid comprise between about 25 and about 250 nucleotides, with recombination regions of about 25 to about 60 nucleotide base pairs being particularly preferred.

The target nucleic acid (or its various component fragments) incorporated into an expression vector according to the invention can be of any size, although sizes ranging from about 300 nucleotides to up to about 1 million nucleotide base pairs are preferred. Such target nucleic acids may include one or more genes and/or their associated regulatory regions. These target nucleic acids can be derived from any source, for example, from genomic sources or from cDNA libraries, including tissue-specific, normalized, and subtractive cDNA libraries. Genomic sources include the genomes (or fragments thereof) of various organisms, including pathogenic organisms such as viruses (e.g, HIV and hepatitis viruses) and cellular pathogens. Moreover, target nucleic acids can be obtained from any organism, including any plant or any animal, be they eukaryotic or prokaryotic. In certain embodiments, a target nucleic acid encodes a gene which is a disease-associated gene, ie., the presence, absence, expression, lack of expression, altered level of expression, or existence of an altered form of which correlates with or causes a disease.

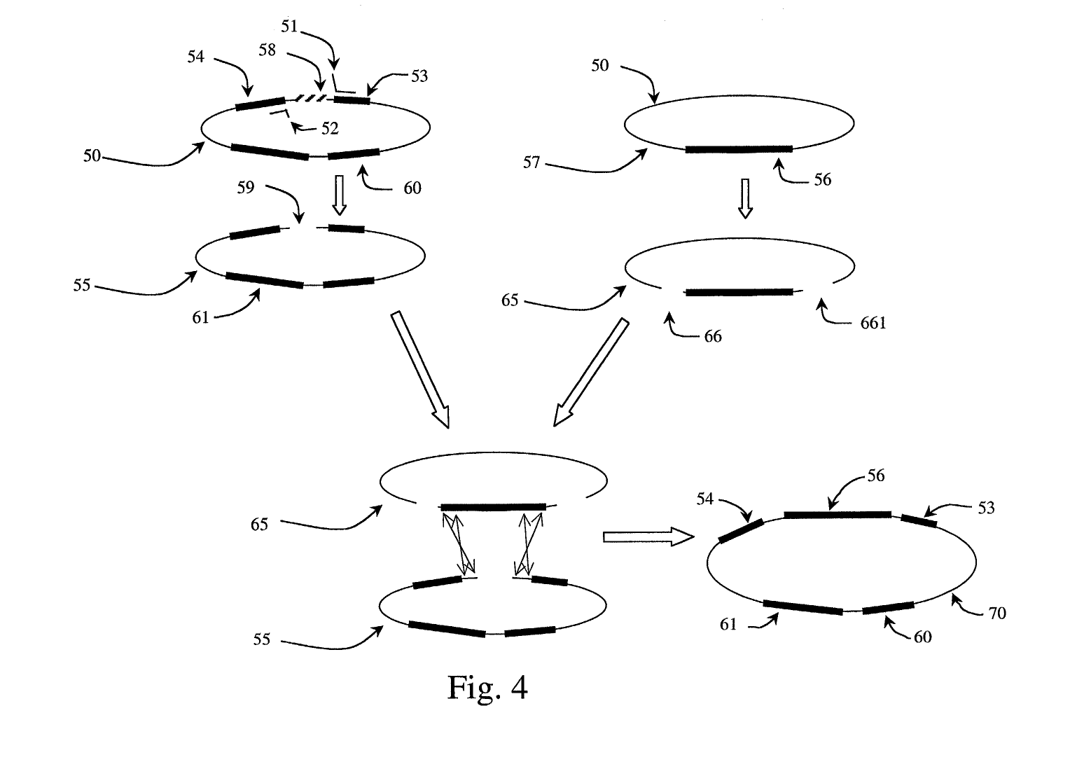

FIG. 4 depicts construction of a human cDNA-containing yeast expression vector prepared using gap repair with an inverse PCR-amplified plasmid (GRIPP™) technology, wherein base vector 50 (which contains an APS 60 such as the yeast 2μ sequence, and a selection marker 61 such as LEU2) is amplified by inverse PCR using two primers 51 and 52, each of which contains a 5′ portion coding for a 45 nucleotide sequence-specific recombination region (homologous recombination site) and a 3′ priming portion (indicated by being parallel to the primer binding regions of the base vector 50. The inverse PCR step produces a linear expression vector intermediate having sequence-specific recombination regions at both termini. A desired target polynucleotide 56 (for example, a human cDNA) carried on an E. coli vector 57 is linearized by cleaving or otherwise introducing one or more double-stranded breaks 66 and/or 661 in the vector portion of the E. coli vector (preferably outside the human cDNA in this embodiment), producing a linear nucleic acid molecule 57 containing a contiguous pre-recombination nucleotide sequence. Alternatively, the nucleic acids can be provided in circular form and transformed into a host cell that expresses restriction endonucleases capable of cleaving the nucleic acids at the desired locations. The linear nucleic acid molecule 57 and the expression vector intermediate 55 are then introduced into a recombination-competent yeast, whereupon homologous recombination occurs between the sequence-specific recombination regions of the expression vector intermediate and the corresponding sequence-specific recombination regions of the target nucleic acid to produce an expression vector 70. For example, the base vector can be GAL-pARC and the target nucleic acid can be the human cyclin A1 gene present as a cDNA. As illustrated, the target nucleic acid can be provided as an isolated HindIII-SacI fragment, or as part of a larger linear nucleic acid molecule. This experiment is described in detail in Example 1 below. Amplification of the first polynucleotide (vector precursor) in this manner permits one to adapt any useful vector to the method of the invention without requiring in vitro ligation, simply by providing appropriate primers and amplifying the vector by inverse PCR. This also permits facile construction of vector precursors having any desired homologous recombination sequence.

One can apply the same principles to preparation of the target polynucleotide. For example, a cDNA library can be provided as a plurality of sequences, each inserted in a standard vector (and having common flanking sequences). The target sequences are amplified using primers comprising a homologous recombination sequence and a sequence complementary to a common sequence of the standard vector, resulting in a plurality of polynucleotides having homologous recombination sequences flanking a plurality of target genes, which can be of unknown sequence. Entire cDNA libraries can be prepared and transformed in this fashion, for example to prepare a library of surrogate genetics host cells. Alternatively, one can selectively amplify desired target sequences by employing one or more primers that hybridize only to those sequences that are of interest. For example, one can employ a first primer that hybridizes to the standard vector sequence, and thus does not distinguish between target genes, and one or more second primers that are complementary to a sequence common to the target genes of interest, such as a zinc finger motif or a protease domain.

Further, one can apply the method of the invention to generate diverse libraries of previously unknown polypeptides. In this embodiment, the target nucleic acid comprises a mixture of different non-contiguous polynucleotides, each of which comprises a sequence encoding a protein domain or fragment (for example, a protease domain, an immunoglobulin fold, a cytokine binding region, a zinc finger, and the like) flanked by homologous recombination sequences capable of recombination with each other. When applied to the method of the invention, this results in a plurality of different polypeptides having different numbers of domains, different included domains, and domains in a variety of different orders. Certain features can be required by judicious selection of homologous recombination sequences. For example, one can generate membrane-bound surface proteins by including a membrane translocation signal sequence in all of the target sequences that carry the first homologous recombination site (i.e., one of the two sites capable of recombining with the base vector precursor), and a membrane anchor in all of the target sequences that carry the last homologous recombination site (i.e., the other of the two sites capable of recombining with the base vector): thus regardless of which sequences are inserted between the first and last domain, the polypeptide resulting from expression of the vector will include a membrane translocation signal and a membrane anchor. For example, the signal sequence can be flanked by a first and a third recombination sequence, and the anchor flanked by a third and second recombination sequence, with a variety of different sequences flanked by two third recombination sequences. These domains can further comprise splicing signals situated between the target sequences and their flanking homologous recombination sequences, effectively forming combinatorial exons.

Another aspect of the invention relates to methods of making expression vector intermediates useful in the practice of this invention. In such methods, a base vector comprising an autonomous propagation sequence, a first primer binding sequence, and a second primer binding sequence is amplified using at least a first primer and a second primer. The first primer typically comprises of 5′ portion having a first sequence-specific recombination sequence and a 3′ portion having a priming portion substantially complementary (i.e., having sufficient complementarity to enable amplification of the desired nucleic acids but not other, undesired molecules) to the first primer binding sequence of the base vector. Similarly, the second primer comprises a 5′ portion having a second sequence-specific recombination sequence and a 3′ portion having a priming portion substantially complimentary to the second primer binding sequence of the base vector. Amplification of the base vector (which can be either linear or circular prior to initiation of the amplification process) results in the production of a linear expression vector intermediate having a first terminus comprising a first sequence-specific recombination region and a second terminus comprising a second sequence-specific recombination region. In certain embodiments, the base vector is a plasmid, particularly a plasmid such as are known in the art and which are based on various bacterial- or yeast-derived extra-chromosomal elements. In certain other embodiments of the invention, the base vector further comprises one or more selectable markers, transcription initiation sequences, and/or transcription termination sequences. As those in the art will appreciate, elements intended to regulate expression of genes carried in the target nucleic acid should be positioned in the expression vector so as to be functionally or operably associated with the gene(s) to be expressed. The particular positioning of such elements depends upon those elements employed, the host cell, the gene(s) to be expressed, and other factors known in the art. As a result, the final design of a particular expression vector made in accordance with the instant teachings is a matter of choice and depends upon the specific application.

Yet other aspects of the invention concern expression vector intermediates made in accordance with the foregoing methods, and host cells containing the same. Still another aspect of the invention relates to methods of making multiple distinct expression vector intermediates useful in the practice of the present invention. In such methods, a base vector is amplified to generate two or more expression vector intermediates each having unique sequence-specific recombination regions which allow for homologous recombination with different target nucleic acids. Such amplification reactions are preferably carried in separate reaction mixtures to produce distinct expression vector intermediates. In particularly preferred embodiments of such a high throughput approach, the requisite manipulations are performed in an automated fashion wherein one or more steps is performed by a computer-controlled device.

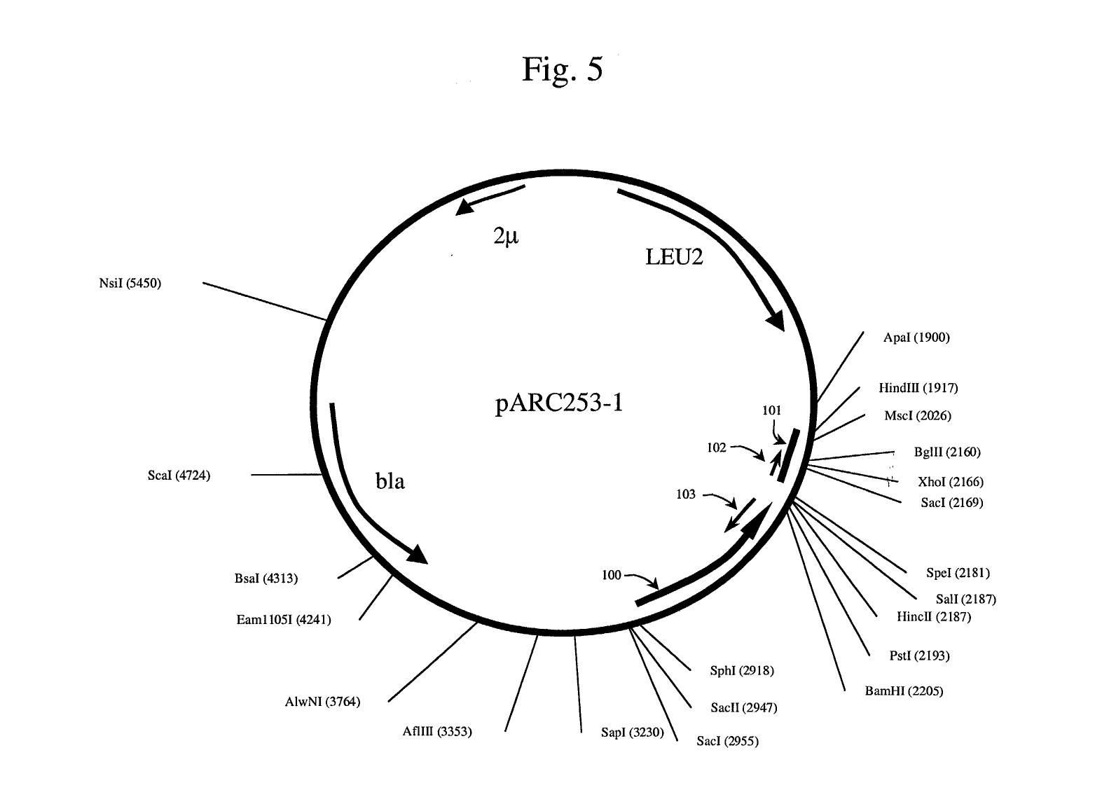

FIG. 5 shows a map of base vector pARC 253-1. This base vector comprises a yeast 2μ autonomous propagation sequence, a LEU2 selectable marker, a GAL1 promoter, a GAL4 terminator, and two primer binding sites. A number of restriction site locations are also provided, as are the vector annealing sequences of two primers which can be used to amplify this base vector.

FIG. 6 illustrates an embodiment of a “Triple GRIPP” procedure where two PCR reactions produce two amplification constructs which, upon host cell-mediated recombination, result in the expression vector intermediate component of the human cDNA expression vector. As shown in the figure, the plasmid pSTU201 comprises a Kluyveromyces lactis URA3 expression cassette. The central lightly shaded area designates the URA3 coding region, which is flanked on the left by its promoter (20) and on the right by its 3′ untranslated region. Two primers, the “Universal 20-mer” and the “Specific 40/20-mer” are used to amplify this cassette. The Specific 40/20-mer has two regions, a 3′ domain comprising a 20 nucleotide priming portion substantially complementary to the corresponding primer binding region on pSTU201 and a 40 nucleotide 5′ domain that lacks complementarity with the plasmid but provides the homology needed for the later in vivo recombination step. The second PCR reaction, PCR B, involves amplification of plasmid pARC 243-1 using Specific 40/20-mer and Universal 40/20-mer, each of which comprises a 5′ 40 nucleotide portion encoding a region for homologous recombination and a 20 nucleotide 3′ priming portion complementary to the corresponding primer binding site on the pARC vector. The shaded “P” region designates an inducible promoter region in the pARC vector just upstream from a multiple cloning site (“MCS”) and a GAL4 terminator sequence (second shaded region on pARC base vector). The human cDNA (shaded regions represent the open reading frame flanked by 5′ and 3′ untranslated sequences) to be recombined into the expression vector is carried on a bacterial vector and is bounded at its 5′ and 3′ ends by restriction sites X and Y. After digestion with the appropriate restriction enzymes and isolation of the human cDNA insert, the cDNA fragment is co-transformed into an appropriate yeast host cell with the amplified K. lactis URA3 cassette expression vector intermediate fragment and the pARC expression vector intermediate fragment.

Other features and advantages of the invention will be apparent from the figures, examples, and claims.

### EXAMPLES

Materials and methods used throughout the examples are first written below, and the examples that follow will serve to further illustrate various aspects of the present invention and are not intended to act in any manner as limitations on the claimed invention. Standard techniques in molecular biology are provided in texts such as Current Protocols in Molecular Biology, eds. Ausubel, et al., John Wiley & Sons, Inc. (1995; ISBN 0-471-50338-X), and Molecular Cloning, A Laboratory Manual, 2nd ed., eds. Sambrook, et al., Cold Spring Harbor Laboratory Press (1989; ISBN 0-87969-309-6)

Yeast Transformation Protocol.

Yeast used to produce expression vectors according to the invention were transformed as follows: A 50 mL overnight culture of yeast strain YST 134 (derived from parent strain YST112 and having the following genetic classification: MATa; ade2-101, his3Δ200; leu2-3, 112, trp1-1; ura3-52; cyh2) was grown at 37° C. in YPD medium (final concentration: 1% yeast extract; 2% peptone; 2% dextrose) to an OD₆₀₀ of 1.0-1.5. Cells were pelleted by spinning the culture at 3,000 rpm for 3 min. in a clinical centrifuge. The cell pellet was washed twice with 25 mL of 100 mM lithium acetate (LiAc) (prepared fresh from a filter-sterilized stock solution containing 102 g LiAc/L) per wash. The pelleted cells can be stored up to four days at 4° C., although freshly prepared cells are preferred.

The pelleted, transformation-competent cells were then transferred to a 1.5 mL Eppendorf tube and resuspended in 500 μL 100 mM LiAc. For each transformation, 50 μL of resuspended cells were combined with 5 μL of single stranded carrier DNA (10 mg/mL fish DNA (Boehringer Mannheim) freshly boiled and quenched on ice), 50-100 ng of the appropriate amplified expression vector intermediate, and 100-500 ng of target nucleic acid-containing DNA and left standing at room temperature. After a 15 min. incubation, 300 μL of LiAc/PEG (12 mL PEG-3350 (15 g of PEG-3350 (Sigma Chemical Co.) dissolved in 16.5 mL of water, filter sterilized, and stored frozen) dissolved in 1.5 mL 1 M LiAc and 1.5 mL water) was added. Following a 30-90 min. room temperature incubation, 38 μL of DMSO was added to each transformation reaction and transferred to a 42° C. bath for 15 min. Cells were then pelleted for 45 sec at 2,000 rpm in a clinical centrifuge, washed once with 1 mL sterile water, and immediately plated onto yeast synthetic dropout plates previously prepared by combining 1.7 g yeast nitrogen base (without amino acids or ammonium sulfate), 5.0 g ammonium sulfate, 2.0 g “Dropout” powder (lacking Trp or Leu, depending on the selection capability conferred by the employed; purchased from BIO101 (La Jolla, Calif.)), and 20 g agar in 900 mL of water, followed by autoclaving. After being allowed to cool slightly, 50 mL of a 20% filter-sterilized glucose or galactose stock solution was added, after which aliquots were dispensed to plastic petri dishes and allowed to solidify. Plates lacking leucine were labeled “Glucose SC-Leu,” while those lacking tryptophan were labeled “Glucose SC-Trp.”

### Example 1

### Production of Human Cyclin A-Containing Expression Vector Using GRIPP

A yeast expression vector containing the human cyclin A gene under the control of a GAL-inducible promoter was produced in an expression vector intermediate using GRIPP (gap repair with an inverse PCR-amplified plasmid). The production of this GRIPP expression vector is diagrammatically illustrated in FIG. 4. The base vector used in this example, pARC 253-1 (see FIG. 5), contains a GAL1 promoter (Mol. Cell. Biol. (1984) 4:1985-90; GenBank accession number K02115) and GAL4 transcription termination sequence (Mol. Cell. Biol. (1984) 4:260-67; GenBank accession number K01486). Selection capability was conferred by the LEU2 open reading frame carried on the base vector. 45 base pair “hooks,” or regions of homology with the 5′ and 3′ ends of the human cyclin A1 gene, were included at the 5′-termini of the primers used to inverse-PCR amplify pARC 253-1, as described below, to produce the expression vector intermediate co-transformed with the human cyclin A coding region into recombination-capable; competent yeast. The human cyclin A1 gene is toxic to yeast cell growth, and thus expression vectors which correctly recombined to contain the human cyclin Al gene exhibited a detectable phenotype, ie., cell death in the presence of the inducer galactose and cell growth when expression from the GAL1 promoter was repressed due to the absence of the inducer from the growth media.

Initially, a bacterial plasmid vector (pBlueScript II SK⁺; Stratagene, Inc., San Diego, Calif.) containing a human cyclin A1 cDNA clone (GenBank accession no. U97680) was cleaved with HindIII and SacI to release the 1.1 kb human cDNA, followed by purification using the QIAEXII K7 from Quiagen (Valencia, Calif.). SacI- and HindIII-only digests of the bacterial vector were also performed, linearizing the plasmid, followed by purification.

The human cyclin A1 gene (either as a HindIII-SacI fragment or as SacI or HindIII digests of the human cyclin A1 gene-containing plasmid) was then co-transformed with an amplified expression vector intermediate prepared as follows: 1 μL (about 10 ng) of a 1:100 dilution of a 1 μg/mL solution of the base vector pARC 253-1 (see FIG. 3) was added to each of four 1.5 mL Eppendorf tubes containing 5 μL 10×PCR buffer, 1 μL of freshly prepared 10 mM dNTPs (10 mM of each dATP, dCTP, dTTP, and dGTP), 1 μL (10 pmo/μL) of each of primers 1 and 2 (SEQ ID NOS:1 and 2, respectively), 42 μL water, and 0.5 μL Stratagene Taq(+) DNA polymerase. Primers 1 and 2 had the following nucleotide sequences:

Primer 1 (SEQ ID NO:1):
5′-TGTGTGTCCCTCATGGAGCCACCTGCAGTTCTTCTTCTACAATAAGAGATCTATGAATCGTAGATACTG-3′
Primer 2 (SEQ ID NO:2):
5′-CCTGATTCTTGTACTCGACCCAACCTTTCTCTTCTTCTTTGGCATtatattCCTTGACGTTAAAGTATAGAG-
GTATATTAAC-3′

The underlined sequences in primers 1 and 2 represent the priming portions of the respective primers, which priming portions hybridized to complementary nucleotide sequences on pARC 253-1 during the annealing steps of PCR. The six nucleotide “tatatt” sequence of primer 2 represents a Kozak sequence.

The pARC 253-1-based expression vector intermediate was then prepared by heating each of the reactions to 95° C. for 3 min. to denature the double-stranded base vector. Thereafter, 33 cycles of denaturation (94° C. for 1 min.), annealing (58° C. for 45 sec.), and extension (72° C. for 9 min.) were then performed in a thermocycler with a heated lid to prevent evaporation, with the final extension step being allowed to proceed for an additional 20 min. to fill in the ends of the PCR products.

After completing the PCR reactions, the four reactions were pooled and a 2 μL aliquot of the pooled reactions was removed, diluted in loading buffer, and run on a 0.7% ethidium bromide-stained agarose gel against standards known to contain particular concentrations of DNA in order to quantitate the amount of PCR reaction products generated. About 50 ng of the expression vector intermediate was then used to conduct each transformation with competent yeast YST 134 in accordance with the transformation protocol described above. Three transformations were performed, with one being a no-insert control (the amplified expression vector intermediate only). The other two transformations involved the co-introduction of the expression vector intermediate and either the HindIII-SacI human cyclin A1 gene-containing fragment or the SacI-only digest of pTP9. In each transformation (except the vector-only control), about 50 ng of the amplified expression vector intermediate was co-transformed with approximately 100 ng of insert DNA. Clones containing the desired human cyclin A1 cDNA expression vector constructs were plated “Dropout” plates lacking leucine (i e., Glucose SC-Leu plates), thereby enabling selection of yeast containing the recombined expression vector.

To confirm the presence of the human cyclin cDNA, colonies from the Glucose SC-Leu plates were replica-plated onto plates containing 2% (weight/volume) galactose instead of glucose to induce expression of the human cyclin A1 cDNA, if present, from the GAL1 promoter. An additional replica-plating control was also performed using Glucose SC-Leu plates. The table below shows the numbers of viable colonies detected.

TABLE 1

| | Number of | | |
|---|---|---|---|
| DNA(s) | Colonies | Number of Colonies | % of Correct |
| Transformed | Glucose SC-Leu | Galactose SC-Leu | Clones |
| EVI* only | 15 | 15 | 0% |
| EVI + cDNA** | 209 | 5 | 97% |
| EVI + L.P.*** | 179 | 8 | 95% |

*The expression vector intermediate only
**The expression vector intermediate and the HindIII/SacI human cyclin A1 cDNA fragment
***The expression vector intermediate and the SacI-digested human cyclin A1 cDNA-containing plasmid

These results show that 95% or more of the clones produced by GRIPP correctly recombined, irrespective of whether the human cyclin A1 cDNA was recombined from a short fragment or from a linearized bacterial plasmid containing the cDNA.

### Example 2

### Production of Human Cyclin A1-Containing Expression Vector Using “Triple” GRIPP

This example also describes the production of a yeast expression vector containing a human cyclin A1 gene regulated by a GAL1 promoter (see FIG. 6). In contrast to the methods used in Example 1, wherein two nucleic acid molecules, an expression vector intermediate and a nucleic acid carrying the human cyclin A1 cDNA, were co-transformed into competent yeast, the GRIPP procedure described herein uses recombination between three different nucleic acid molecules to form the human cyclin A1 expression vector, and is thus referred to as a “triple” GRIPP procedure. In this embodiment of triple GRIPP, two of the three nucleic acid molecules comprise different parts of the expression vector intermediate, and below each is referred to as an “expression vector intermediate fragment”.

The human cyclin A1 gene used in this example was prepared as described in Example 1 above, and freshly prepared competent cells of yeast strain YST 134 were again used. For the triple GRIPP method, two PCR reactions were performed to produce the expression vector intermediate fragments. PCR reaction A (“PCR A”) amplified a Kluyveromyces lactis URA3 cassette from plasmid pSTU201 (see FIG. 6) using two primers, KL URA3 Universal and Cyclin-KL URA3, the sequences of which appear below:

KL URA3 Universal (SEQ ID NO:3):
5′-TTAATGGGGAGCGCTGATTC-3′
Cyclin-KL URA3 (SEQ ID NO:4):
5′-TGTGTGTCCCTCATGGAGCCACCTGCAGTTCTTCTTCTACAATAAgatcgttttatttaggttctatcgag-3′

In these two primers, the priming portions are underlined. In the Cyclin-KL URA3 primer, the 45 5′ nucleotides code for the region in which homologous recombination can occur.

Four separate PCR amplification reactions were performed. In each amplification, 10 pmol of each these primers (each in 1 μL) were combined with 5 μL of 10×Taq(+) PCR buffer, 5 μL of a solution containing 2 mM of each of dATP, dCTP, dTTP, and dGTP, 0.5 μL of Taq(+) DNA polymerase, 30.5 μL water, and 1 μL of a 1:20 dilution of a 1 mg/mL solution of pSTU201. The reactions were first heated to 95° C. for 3 min. 33 PCR cycles were then performed. Each cycle involved 1 min. denaturation at 94° C., 45 sec. annealing at 58° C., and 3 min. of extension at 72° C. The final extension step was allowed to continue for an additional 20 min. to ensure that the ends of the PCR products were filled in. After the reactions were completed, they were pooled.

A second set of four PCR amplifications was also conducted using the same conditions, except that two different primers, Cyclin-GALpro and KL URA3-GALterm Uni, and 1 μL of a 1:100 dilution of a 1 mg/mL solution of pARC were used in the reactions. The nucleotide sequences of the Cyclin-GALpro and KL URA3-GALterm Uni primers appears below:

Cyclin-GALpro (SEQ ID NO:5):
5′-CCTGATTCTTGTACTCGACCCAACCTTTCTCTTCTTCTTTGGCATtatattCCTTGACGTTAAAGTATAGAGGTATATTAAC-3′
KL URA3-GALterm Uni (SEQ ID NO:6):
5′-CTGGATGGGAAGCGTACCAAAAGAGAATCAGCGCTCCCCATTAAgagatctatgaatcgtagatactg-3′

In the Cyclin-GALpro primer, the “tatatt” sequence is a Kozak sequence. The priming portion of each of the primers is underlined. The 45 5′ nucleotides of the Cyclin-GALpro primer code for the region of homology to be incorporated into the vector with respect to the region just 3′ to the start codon of the human cyclin A1 coding region, whereas the 44 5′-most nucleotides of the KL URA3-GALterm primer provide sequence homology between the pARC expression vector intermediate fragment and the 3′ region of the K. lactis URA3 cassette.

After amplifying the expression vector intermediate fragments, approximately 200 ng of each (quantitated as described above) were used, along-with about 200 ng of a human cyclin A1 cDNA construct (either a gel-purified HindIII-SacI fragment or a HindIII- or SacI-only digest of pTP9) to co-transform freshly prepared, competent cells of yeast strain YST 134 in ten transformation experiments, as described in Table 2 below. Four one-quarter volume aliquots of each transformation were then plated on selective media containing glucose as the carbon source and lacking either tryptophan or uracil. In Table 2, “V” designates amplified pARC, “U” designates the amplified K. lactis URA3 cassette, “H/S-I” refers to the HindIII-SacI human cyclin cDNA insert, “HcDNA” refers to pTP9 linearized with HindIII, and “ScDNA” refers to pTP9 linearized with SacI.

TABLE 2

| DNA | Colonies Glu | Colonies Gal | % Correct | Colonies | Colonies Gal | % Correct |
|---|---|---|---|---|---|---|
| transformed | SC-Leu | SC-Leu | clones | Glu SC-Ura | SC-Ura | colonies |
| V | 2 | 1 | 50 | 0 | 0 | 0 |
| V + URA | 13 | 13 | 0 | 7 | 7 | 0 |
| V + ScDNA | NA | NA | NA | NA | NA | NA |
| V + HcDNA | 10 | 9 | 10 | 0 | 0 | 0 |
| V + H/S − I | 12 | 6 | 50 | 0 | 0 | 0 |
| V + URA + S | 159 | 135 | 15 | 41 | 0 | 100 |
| V + URA + H | 66 | 37 | 44 | 33 | 12 | 63 |
| V + URA + H/S − I | 167 | 27 | 84 | 143 | 7 | 95 |
| No DNA | 0 | 0 | 0 | 0 | 0 | 0 |
| pARC only | 106 | 106 | 0 | 0 | 0 | 0 |

These results demonstrated that Triple GRIPP procedures can be used to generate desired expression vectors.

While embodiments and applications of the present invention have been described in some detail by way of illustration and example for purposes of clarity and understanding, it would be apparent to those individuals whom are skilled within the relevant art that many additional modifications would be possible without departing from the inventive concepts contained herein. The invention, therefore, is not to be restricted in any manner except in the spirit of the appended claims.

It is understood that any patent, patent application, text, scientific article, or other reference mentioned above is not admitted to be prior art, and that all references are hereby incorporated by reference in their entirety. Any term used in the singular shall include the plural, and vice versa, unless the context dictates otherwise. Terms shall be understood to be inclusive, for example, “including” means “including, without limitation.”

What is claimed:

1. A method for generating a recombinant vector, comprising:
a) Providing a first nucleic acid comprising a first homologous recombination sequence and a second homologous recombination sequence; and a target nucleic acid comprising a target sequence and homologous recombination sequences homologous to said first and second homologous recombination sequences; wherein at least one of said nucleic acids comprises an autonomous propagation sequence, wherein said first and second homologous recombination sequences are not substantially homologous to each other;
b) Transforming a recombination-competent host cell with said first nucleic acid and said target nucleic acid; and
c) Allowing said host cell to generate said recombinant vector comprising portions of said first nucleic acid and said target nucleic acid and said autonomous propagation sequence through homologous recombination.

2. The method of claim 1.), wherein said target nucleic acid is provided by:
a) Providing a target precursor polynucleotide comprising said target sequence, and first and second primer binding sites upstream and downstream of said target sequence;
b) Amplifying said target precursor polynucleotide using first and second primers, wherein said first primer comprises a sequence complementary to said first primer binding site and a first homologous recombination sequence, and said second primer comprises a sequence complementary to said second primer binding site and a second homologous recombination sequence.

3. The method of claim 1.), wherein said first nucleic acid is provided by:
a) Providing a vector precursor polynucleotide comprising an autonomous propagation sequence, and first and second primer binding sites upstream and downstream of said autonomous propagation sequence;
b) Amplifying said target precursor polynucleotide using first and second primers, wherein said first primer comprises a sequence complementary to said first primer binding site and a first homologous recombination sequence, and said second primer comprises a sequence complementary to said second primer binding site and a second homologous recombination sequence.

4. The method of claim 1.), wherein said target nucleic acid comprises a plurality of non-contiguous nucleic acids, each separate nucleic acid comprising two homologous recombination sites, wherein each homologous recombination site is homologous to a homologous recombination site of said first nucleic acid or of another separate nucleic acid.

5. The method of claim 1.), wherein said target nucleic acid comprises a plurality of different contiguous nucleic acids, each different contiguous nucleic acid comprising a different target gene, wherein said method provides a plurality of different vectors.

6. The method of claim 1.), wherein said first nucleic acid comprises an autonomous propagation sequence.

7. The method of claim 1.), wherein said first nucleic acid comprises a negative selectable marker, and said negative selectable marker is positioned between said first and second homologous recombination sites, and wherein said negative selectable marker is excised during homologous recombination.

8. The method of claim 1.), wherein said first nucleic acid further comprises a first positive selectable marker.

9. The method of claim 1.), wherein said target nucleic acid further comprises a target positive selectable marker.

10. The method of claim 9.), wherein said first nucleic acid further comprises a first positive selectable marker, wherein said first positive selectable marker is distinguishable from said target positive selectable marker.

11. The method of claim 1.), wherein said host cell comprises a yeast cell.

12. The method of claim 1.), wherein said host cell comprises a bacterium.

13. The method of claim 1.), wherein said host cell comprises a mammalian cell.

14. The method of claim 1.), wherein said host cell comprises a plant cell.

15. The method of claim 1.), wherein said first nucleic acid comprises a promoter operable in said host cell.

16. The method of claim 1.), wherein providing said target nucleic acid comprises:
a) Providing a plasmid comprising said target sequence and flanking primer binding sequences;
b) Providing primers complementary to said primer binding sequences, said primers being capable of initiating a PCR amplification; and
c) Amplifying said nucleic acid by PCR.

17. The method of claim 16.), wherein said primers further comprise homologous recombination sequences.

18. The method of claim 16.), wherein said PCR amplification comprises inverse PCR.

19. The method of claim 16.), wherein said target sequence comprises a first DNA molecule comprising a coding sequence and a homologous recombination sequence, and a second DNA molecule comprising a heterologous sequence and a homologous recombination sequence homologous to said first DNA molecule homologous recombination sequence, wherein said first DNA molecule comprises an additional homologous recombination sequence.

20. The method of claim 1.), wherein said target nucleic acid comprises a plurality of different non-contiguous polynucleotides, each non-contiguous polynucleotide comprising a target sequence encoding a protein fragment flanked by homologous recombination sequences, wherein said different non-contiguous polynucleotides can assemble in a plurality of different orders.

21. The method of claim 20.), wherein a target nucleic acid further comprises a splice sequence between said target sequence and each flanking homologous recombination sequence.

22. A kit for preparing vector precursors for use with a given nucleic acid comprising a target sequence, said kit comprising
a) A vector precursor polynucleotide comprising an autonomous propagation sequence and first and second homologous recombination sequences;
b) A pair of primers, each primer comprising a homologous recombination sequence and a sequence complementary to said given nucleic acid, wherein amplification of said given nucleic acid with said primers provides a target nucleic acid comprising said target sequence flanked by first and second homologous recombination sequences.
